SEQUENCE LISTING (37 sequences)

SEQ ID NO: 1 - protein, 16 aa, Artificial Sequence
  Polycationic nucleic acid binding component
KKKKKKKKKKKKKKKK

SEQ ID NO: 2 - protein, 4 aa, Artificial Sequence
  Cleavable portion of spacer element peptide
  MISC_FEATURE (2)..(2): The residue at this position may be K or V
RKKR

SEQ ID NO: 3 - protein, 4 aa, Artificial Sequence
  Cleavable portion of spacer element peptide
  MISC_FEATURE (2)..(2): The residue at this position may be K or V
RKRR

SEQ ID NO: 4 - protein, 4 aa, Artificial Sequence
  Cleavable portion of spacer element peptide
  MISC_FEATURE (3)..(3): The residue at this position may be F, I, L, V or Y, preferably F, L or Y
  MISC_FEATURE (4)..(4): The residue at this position may be A, E or G
GFFA

SEQ ID NO: 5 - protein, 4 aa, Artificial Sequence
  Cleavable portion of spacer element peptide
GFLG

SEQ ID NO: 6 - protein, 5 aa, Artificial Sequence
  Linker portion of spacer element peptide
  misc_feature (1)..(1): The residue at this position can be any naturally occuring amino acid
  misc_feature (3)..(3): The residue at this position can be any naturally occuring amino acid
ASAGG

SEQ ID NO: 7 - protein, 5 aa, Artificial Sequence
  Linker portion of spacer element peptide
  misc_feature (1)..(1): The residue at this position can be any naturally occuring amino acid
  misc_feature (3)..(3): The residue at this position can be any naturally occuring amino acid
ASAGA

SEQ ID NO: 8 - protein, 7 aa, Artificial Sequence
  Cell surface receptor binding component
RRETAWA

SEQ ID NO: 9 - protein, 4 aa, Artificial Sequence
  Cell surface receptor binding component
  MISC_FEATURE (2)..(2): The residue at this position is P or Q, preferably P
LPHK

SEQ ID NO: 10 - protein, 7 aa, Artificial Sequence
  Cell surface receptor binding component
  MISC_FEATURE (4)..(4): The residue at this position is A or T, preferably A
PSGAARA

SEQ ID NO: 11 - protein, 7 aa, Artificial Sequence
  Cell surface receptor binding component
  MISC_FEATURE (2)..(2): The residue at this position may be any acidic amino acid residue, preferably E or Q
  MISC_FEATURE (2)..(2): The residue at this position may be any acidic amino acid residue, preferably E or Q, more preferably E
SERSMNF

SEQ ID NO: 12 - protein, 7 aa, Artificial Sequence
  Cell surface receptor binding component
  MISC_FEATURE (2)..(2): The residue at this position may be P or Q, preferably P
LPHKSMP

SEQ ID NO: 13 - protein, 7 aa, Artificial Sequence
  Cell surface receptor binding component
PSGAARA

SEQ ID NO: 14 - protein, 7 aa, Artificial Sequence
  Cell surface receptor binding component
LQHKSMP

SEQ ID NO: 15 - protein, 7 aa, Artificial Sequence
  Cell surface receptor binding component
LPHKSMP

SEQ ID NO: 16 - protein, 7 aa, Artificial Sequence
  Cell surface receptor binding component
VKSMVTH

SEQ ID NO: 17 - protein, 7 aa, Artificial Sequence
  Cell surface receptor binding component
SERSMNF

SEQ ID NO: 18 - protein, 7 aa, Artificial Sequence
  Cell surface receptor binding component
VGLPHKF

SEQ ID NO: 19 - protein, 7 aa, Artificial Sequence
  Cell surface receptor binding component
YGLPHKF

SEQ ID NO: 20 - protein, 7 aa, Artificial Sequence
  Cell surface receptor binding component
PSGAARA

SEQ ID NO: 21 - protein, 7 aa, Artificial Sequence
  Cell surface receptor binding component
SQRSMNF

SEQ ID NO: 22 - protein, 7 aa, Artificial Sequence
  Cell surface receptor binding component
PSGTARA

SEQ ID NO: 23 - protein, 5 aa, Artificial Sequence
  Cell surface receptor binding component
  MISC_FEATURE (2)..(2): The residue at this position can be any naturally occuring amino acid
  MISC_FEATURE (3)..(3): The residue at this position can be any naturally occuring amino acid
  MISC_FEATURE (4)..(4): The residue at this position can be any naturally occuring amino acid
PAAAT

SEQ ID NO: 24 - protein, 4 aa, Artificial Sequence
  Cell surface receptor binding component
  MISC_FEATURE (3)..(3): The residue at this position can be any naturally occuring amino acid
PSAS

SEQ ID NO: 25 - protein, 5 aa, Artificial Sequence
  Cell surface receptor binding component
  misc_feature (2)..(2): The residue at this position can be any naturally occuring amino acid
  misc_feature (3)..(3): The residue at this position may be any amino acid with an amide side chain, such as N or Q
  misc_feature (4)..(4): The residue at this position can be any naturally occuring amino acid
QANAQ

SEQ ID NO: 26 - protein, 7 aa, Artificial Sequence
  Cell surface receptor binding component
CRGDCLG

SEQ ID NO: 27 - protein, 7 aa, Artificial Sequence
  Cell surface receptor binding component
CRGDCLG

SEQ ID NO: 28 - protein, 11 aa, Artificial Sequence
  Cell surface receptor binding component
ACDCRGDCFCG

SEQ ID NO: 29 - protein, 9 aa, Artificial Sequence
  Cell surface receptor binding component
CRGDMFGCA

SEQ ID NO: 30 - protein, 10 aa, Artificial Sequence
  Cell surface receptor binding component
CRRETAWACG

SEQ ID NO: 31 - protein, 9 aa, Artificial Sequence
  Cell surface receptor binding component
CRGEMFGCA

SEQ ID NO: 32 - protein, 10 aa, Artificial Sequence
  Cell surface receptor binding component
CSERSMNFCG

SEQ ID NO: 33 - protein, 10 aa, Artificial Sequence
  Cell surface receptor binding component
CYGLPHKFCG

SEQ ID NO: 34 - protein, 10 aa, Artificial Sequence
  Cell surface receptor binding component
CLPHKSMPCG

SEQ ID NO: 35 - protein, 28 aa, Artificial Sequence
  transfection vector
KKKKKKKKKKKKKKKKGACYGLPHKFCG

SEQ ID NO: 36 - protein, 28 aa, Artificial Sequence
  transfection vector
KKKKKKKKKKKKKKKKGACSERSMNFCG

SEQ ID NO: 37 - protein, 4 aa, Artificial Sequence
  Cleavable portion of spacer element peptide
RVRR

(19) United States
(12) Patent Application Publication
(10) Pub. No.: US 2021/0246470 A1
(43) Pub. Date: Aug. 12, 2021

(54) COMPLEX FOR THE DELIVERY OF CAS9 PROTEINS AND GUIDE RNA TO CELLS

(71) Applicant: UCL Business Ltd, London (GB)

(72) Inventors: Stephen Lewis HART, London (GB); Ahmad Mohammed ALDOSSARY, London (GB); Amy WALKER, London (GB)

(21) Appl. No.: 16/973,908

(22) PCT Filed: Mar. 1, 2019
(86) PCT No.: PCT/GB2019/050583

§ 371 Date: Dec. 10, 2020

(30) Foreign Application Priority Data
Mar. 2, 2018 (GB) 1803419.9

(51) Int. Cl. C12N 15/88; C12N 9/22; C12N 15/11; A61K 48/00

(52) U.S. Cl. CPC C12N 15/88; C12N 9/22; C12N 2320/32; A61K 48/0008; C12N 2310/20; C12N 15/11

(57) ABSTRACT

A ribonucleoprotein (RNP) delivery system comprising (a) a cationic lipid; (b) a phospholipid; (c) a peptide of the structure A-B-C in which A is a polycationic nucleic acid-binding component, B is a spacer element comprising two or more amino acid residues, and C is a cell surface receptor binding component; and (d) a ribonucleoprotein (RNP) complex comprising a CRISPR-associated protein and guide RNA, pharmaceutical compositions comprising such a ribonucleoprotein (RNP) delivery system, and methods for the treatment or prophylaxis of a condition caused in a human or in a non-human animal by a defect and/or a deficiency in a gene, or for RNA therapy, or for the treatment of a cancer, which comprises administering the RNP delivery system or pharmaceutical composition to the human or to the non-human animal.

### FIELD OF THE INVENTION

The present invention relates to liposome suitable for delivery of ribonucleoprotein (RNP) complexes, i.e. complexes comprising a CRISPR-associated protein, such as Cas9, and guide RNA to a cell. The invention further relates to ribonucleoprotein (RNP) delivery system for use as non-viral vectors for the delivery of ribonucleoprotein complexes to cells, and the use of such complexes in vivo, for example, in genetic therapy approaches to treatment or prophylaxis, or in an in vitro laboratory setting for gene editing of cells that themselves can be used a therapeutic in regenerative medicine or cancer therapies with engineered immune cells, such as CAR-T cells, or for generating in vivo or in vitro models of disease for genetic research or for screening for drug targets or generating cell-based models of diseases.

### BACKGROUND TO THE INVENTION

Gene delivery for therapy or other purposes is well-known, particularly for the treatment of diseases such as cystic fibrosis and certain cancers. Delivery into a cell of genetic material may be used to correct some deficiency and result in the in vitro production of commercially-useful proteins in so-called cell factories.

Genome editing in which a desired change to the sequence of genomic DNA within a cell has huge potential to provide future treatments for many diseases. A genomic editing tool that efficiently edits any genetic locus with high specificity is highly desirable.

Since 2013, the CRISPR/Cas9 gene editing technique has emerged. The CRISPR/Cas9 technique involves the use of an RNA-guided DNA endonuclease protein (Cas9 nuclease) coupled with guide RNA. The RNA guide localises the RNP complex to the target DNA sequence thus directing the nuclease protein to a specific site. The RNA guide molecule targets a particular genomic DNA sequence, the protospacer adjacent motif (PAM), and binds to it by base pairing.

The CRISPR-associated protein is typically an RNA-guided DNA endonuclease, such as a CRISPR-associated nuclease-9 (Cas9) protein. The Cas9 protein introduces targeted, double stranded breaks in DNA after which the cells natural DNA repair pathways are activated to restore integrity of the chromosome. This may be by the non-homologous end joining repair pathway, an error prone repair process that introduces insertions or deletions (indels) into the chromosome, thus disrupting the reading frame. However, in the presence of a homologous donor DNA molecule, another pathway called homology directed repair, enable targeted insertion and thus correction of the targeted gene. Other variants of the process include engineered nuclease that only nick one strand, or disabled Cas9 that does not make any breaks but carries other enzymatic functions to the targeted region enabling enzymatic modifications such as methylation.

Several naturally occurring or wild type CRISPR-associated nuclease-9 (Cas9) endonuclease proteins have been found to be suitable RNA-guided endonuclease proteins, including SpCas9, a Cas9 protein found in Streptococcus pyrogenes; SaCas9, a Cas9 protein found in Streptococcus aureus; St1Cas9 and St3Cas9, Cas9 proteins found in Streptococcus thermophilus; and NmCas9, a Cas9 protein found in Neisseria meningitides. In addition to Cas9 homologues, other suitable naturally occurring RNA-guided endonuclease proteins include AsCpf1, found in Acidaminococcus sp. and LbCpf1, found in Lachnospiraceae bacterium. Non-natural proteins have also been engineered with particular PAM specificities and to act as fusion proteins without a DNA cutting (i.e. nuclease) function. Examples of engineered nuclease proteins include VQR SpCas9, EQR SpCas9, VRER SpCas9, RHA FnCas9 and KKH SaCas9. Further CRISPR-associated proteins are constantly being discovered and engineered with compatibility with different guide RNA molecules and/or PAM, for example, for greater DNA targeting specificity or for targeting different loci in the genome. Useful CRISPR-associated proteins discovered or engineered to date typically include from 800 to 2000 amino acid residues.

The guide RNA binds to the DNA genome and directs the CRISPR-associated protein, for example Cas9, to the desired portion of the target genome. Some nuclease proteins, including many naturally-occurring Cas9 proteins, use two guide RNA molecules, such as CRISPR-RNA (crRNA) to bind to the protospacer in the DNA target and trans-activating crRNA (tracrRNA) that recruits the Cas9 nuclease protein. The crRNA and tracrRNA molecules generally assemble together into a single non-covalently bound species, together known as the guide RNA (gRNA). However, recently the use of engineered single guide RNA molecules (sgRNA) that have both a covalently linked crRNA and tracrRNA has been adopted for use in many CRISPR/Cas9 systems.

The CRISPR/Cas9 system allows for targeted, specific editing or correction of specific mutations in cells with the potential to disrupt a gene that may be having a deleterious effect, or correcting a gene mutation such as in recessive genetic conditions such as cystic fibrosis or Duchenne muscular dystrophy, to restore expression. There are constantly emerging strategies to use the CRISPR/Cas9 system including base editing, cDNA replacement and many others.

Delivery is a key challenge in developing the therapeutic potential of CRISPR/Cas9. CRISPR/Cas9 can be delivered to cells in several formats including plasmid DNA encoding the CRISPR-associated protein and guide RNA, mRNA encoding the CRISPR-associated protein and guide RNA as synthetic molecules that can include chemically modified bases to enhance activity and stability and reduce toxicity. Finally, CRISPR-associated protein and guide RNAs can be delivered as a preassembled ribonucleoprotein (RNP) complex. The benefit of preassembled RNP delivery over plasmid or mRNA are that there is no need to transcribe RNA or translate protein to elicit editing, which can maximize efficiency, and a shorter term of nuclease activity due to RNP decay leading to greater safety and therefore fewer off-target effects.

It would be desirable to enhance the specificity of RNP complex delivery such that off-target editing is further reduced. In particular, it is desirable to selectively deliver RNP complexes to the required cells. It is also imperative that the transfection efficiency is high with the amount of RNP complex delivered into cells maximised to enhance corrective editing. Another challenge is developing systems that result in high levels of activity over the relatively short timeframe typical for CRISPR/Cas9 systems. Short term activity should reduce off-target effects by activity decaying more rapidly and reducing the risk of Cas9 introducing off-target DNA double strand breaks. An option for delivery of RNP complexes is direct nucleic acid injection, e.g. of Cas9 plasmid DNA or Cas9 mRNA. Whilst it may be possible to use viral vectors to deliver nucleic acid, delivery of RNP complexes requires non-viral methods to be used.

The use of lipid-based transfection complexes (lipoplex) has been used to deliver CRISPR/Cas9 RNPs to cultured cells ex vivo and the Lipofectamine™ CRISPRMAX™ Cas9 in vitro transfection reagent is commercially available. While the composition of the Lipofectamine™ CRISPRMAX™ is not publically available, it is believed to contain a lipid-based transfection reagent and a separate enhancer. That product is said to be effective in transfecting nuclease and guide RNA complexes into a range of eukaryotic cells with good cleavage efficiency in iPSC, mESC, N2A, CHO, A549, HCT116, HeLa, HEK293 cell lines and others. However, the use of lipoplexes to deliver CRISPR/Cas9 RNP complexes to cells in vivo has not been approved and toxicity remains an issue. The use of cationic lipid-based transfection agents to package and deliver RNP complexes has shown some promise in vivo with particular cell types, but the targeting of a broader range of cells remains a problem. However, hard-to-transfect cells (stem cells, neurons, hematopoietic cells, etc.) require more efficient delivery systems such as those based on lentivirus (LVs), adenovirus (AdV) and adeno-associated virus (AAV), which can only deliver plasmids encoding the Cas9 and gRNA, rather than RNP complexes.

There remains a need for a means of efficiently delivering CRISPR/Cas9 RNP complexes in vivo and in vitro targeting particular cell types for transfection.

### SUMMARY OF THE INVENTION

The present invention provides a novel nanoparticle formulation for delivery of RNP comprising a cationic lipid, a phospholipid and a peptide.

Lipopolyplex systems comprising a lipid and an integrin- (or other receptor-) binding peptide have previously used for the delivery of relatively small fragments of nucleic acids as an “LPD” complex comprising (L) lipid, (P) peptide and (D) DNA (or other nucleic acid). LPD complexes achieve transfection via an integrin-mediated or other receptor-mediated pathway; they do not necessarily need to have an overall positive charge so undesirable serum interactions can be reduced. The peptide component provides a nucleic acid packaging function, shielding the DNA or RNA from intracellular or extracellular degradation, endosomal or otherwise. The lipid components mediate interactions with endosomal lipid bilayers by membrane fusion or permeabilisation, reducing endosomal or lysosomal degradation and allowing trafficking of the nucleic acid cargo to the cytoplasm. The peptide component can be designed to be cell-type specific or cell-surface receptor specific. For example, the degree of specificity for integrin or other receptors can confer a degree of cell specificity to the LPD complex.

Specificity results from their targeting to cell-surface receptors (for example integrin receptors), and transfection efficiencies comparable to some adenoviral vectors can be achieved.

While lipopolyplex systems were known to be effective in delivering nucleic acid molecules as LPD complexes, it was not believed that such systems could also be effective in delivering RNPs that include a large nucleaseprotein and a small RNA molecule, the gRNA. Lipopolyplex systems are based on the compaction of genetic material into nanometric particles by electrostatic interaction between the negatively charged phosphate backbone of DNA or RNA and the cationic lipids and peptides. The peptide component provides a nucleic acid packaging function, shielding the DNA or RNA from intracellular or extracellular degradation, endosomal or otherwise. It was not previously known whether the peptide would be effective in shielding the much larger RNP complex, which has a lower density of negative charges than a nucleic acid molecule, and/or whether the CRISPR-associated protein would interact with the peptide in a manner such as to render it ineffective in binding to the integrin or other cell surface receptor.

In a first aspect, the present invention provides a ribonucleoprotein (RNP) delivery system comprising:
(a) a cationic lipid;
(b) a phospholipid;
(c) a peptide of the structure A-B-C in which:
   A is a polycationic nucleic acid-binding component,
   B is a spacer element comprising two or more amino acid residues, and
   C is a cell surface receptor binding component; and
(d) a ribonucleoprotein (RNP) complex comprising a CRISPR-associated protein and guide RNA.

The present inventors were surprised to find that the RNP delivery system of the first aspect of the invention are unexpectedly efficient at RNP delivery. For example, an RNP delivery system of the invention comprising cationic- and phospho-lipids and cell surface receptor targeting peptides assembled together with RNP complexes into nanoparticles have been found to be effective in introducing indels into primary epithelial cells stably expressing GFP, thus silencing the GFP expression, and in Neuro 2A cells. The RNP delivery system of the invention being found to be highly efficient in delivering the RNP complexes to their target cells thus providing augmented corrective editing. As well as being effective in targeting specific cells, it has been found that the RNP delivery system of the invention are more efficient at delivering RNP complexes into cells than, for example, CRISRPMAX™.

In a second aspect, the invention provides a pharmaceutical composition which comprises the ribonucleoprotein (RNP) delivery system of the invention in admixture or conjunction with a pharmaceutically suitable carrier.

In a third aspect, the invention provides the ribonucleoprotein (RNP) delivery system of the invention for use in therapy. In one embodiment, the invention provides the ribonucleoprotein (RNP) delivery system of the first aspect of the invention for use in the treatment or prophylaxis of a condition caused in a human or a non-human animal by a defect and/or a deficiency in a gene, or for RNA therapy. In one embodiment, the invention provides the ribonucleoprotein (RNP) delivery system of the first aspect of the invention for use in the treatment or prophylaxis of cancer in a human or a non-human animal.

In a fourth aspect, the invention provides a method for the treatment or prophylaxis of a condition caused in a human or in a non-human animal by a defect and/or a deficiency in a gene, which comprises administering the ribonucleoprotein (RNP) delivery system of the first aspect of the invention to the human or to the non-human animal. In one embodiment, the invention provides a method for the treatment of a human or non-human animal suffering from a cancer which comprises administering the ribonucleoprotein (RNP) delivery system of the first aspect of the invention to the human or to the non-human animal.

In a fifth aspect, the invention provides the use of the ribonucleoprotein (RNP) delivery system of the first aspect of the invention for the manufacture of a medicament for the treatment or prophylaxis of a condition caused in a human or a non-human animal by a defect and/or a deficiency in a gene, or for mRNA therapy, or for the treatment or prophylaxis of cancer in a human or a non-human animal.

In a sixth aspect, the invention provides a method of making an RNP delivery system of the first aspect of the invention, the method comprising the steps of:

(i) providing a ribonucleoprotein (RNP) complex comprising a CRISPR-associated protein and guide RNA;

(ii) combining the ribonucleoprotein complex from step (i) with lipid component comprising a phospholipid and a cationic lipid; and

(iii) adding a peptide containing (A) a polycationic nucleic acid-binding component, (B) a spacer element comprising two or more amino acid residues and (C) a cell surface receptor binding component to the mixture from step (ii) to form the RNP delivery system.

In a seventh aspect the invention provides a method of editing a gene in mammalian cell, either in vivo or ex-vivo, comprising transfecting the cell with an RNP delivery system of the first aspect of the invention.

### BRIEF DESCRIPTION OF THE FIGURES

FIG. 1: (A) GFP knockdown (%) in NHBE BMI-1 GFP cells following transfection with Cas9 RNP complex using either C14, C16 or C18 DOPE liposomes and peptide Y, at a weight ratio of 1:3:4 RNP:L:P.

FIG. 2: Primary CFBE cells GFP transfection efficiency.

### DETAILED DESCRIPTION OF THE INVENTION

Cationic Lipids (a)

The term “cationic lipid” refers to a lipid comprising at least one fatty acid chain and a polar head group that bears a positive charge. In addition to the positively charges cation, the cationic lipids may comprise a counter anion, for example, an inorganic counter ion, especially a pharmaceutically acceptable anion such as chloride or bromide.

The cationic lipid advantageously comprises a cation the formula (1):

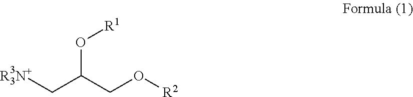

in which R¹ and R² are each independently a C₁₀₋₂₂ unsaturated alkyl chain.

Advantageously, R¹ and R² are are the same or different and are each independently a C₁₀₋₂₂ unsaturated alkenyl group including one or two unsaturated C═C double bonds, preferably one C═C double bond. The C═C double bonds are preferably cis-. Optionally, R¹ and R² are each independently a C₁₂₋₂₀ alkenyl group, for example, a C₁₄₋₁₈ alkenyl group. R¹ and R² optionally have straight (i.e. unbranched) alkyl chains. R¹ and R² may, for example, be each independently selected from C₁₂₋₂₀, straight chain alkenyl groups having one C═C unsaturated double bond; or R¹ and R² may, for example, be each independently selected from C₁₄₋₁₈, straight chain alkenyl groups having one cis-C═C unsaturated double bond. Optionally, R¹ and R² are the same or different and are each independently a straight chain, unsaturated C₁₄, C₁₆ or C₁₈ alkenyl group having one double bond. Optionally R¹ and R² are selected from —(CH₂)₆₋₁₂CH═CH(CH₂)₁₋₉CH₃, especially —(CH₂)₇₋₁₁CH═CH(CH₂)₁₋₈CH₃ or —(CH₂)₈₋₁₀CH═CH(CH₂)₁₋₇CH₃, such as —(CH₂)₁₀CH═CH(CH₂)₁CH₃, —(CH₂)CH═CH(CH₂)₇CH₃ or —(CH₂)₁₀CH═CH(CH₂)₃CH₃. Preferably, the double bond is cis and R¹ and R² are selected from —(CH₂)₆₋₁₂CH[Z]═CH(CH₂)₁₋₉CH₃, especially —(CH₂)₇₋₁₁CH[Z]═CH(CH₂)₁₋₈CH₃ or —(CH₂)₈₋₁₀[Z]CH═CH(CH₂)₁₋₇CH₃, such as —(CH₂)₁₀[Z]CH═CH(CH₂)CH₃, —(CH₂)₈[Z]CH═CH(CH₂)₇CH₃ or —(CH₂)₁₀[Z]CH═CH(CH₂)₃CH₃. Preferably, R¹ and R² are the same.

Each R³ group is independently selected from hydrogen and C₁₋₄ alkyl. Preferably, each R³ is independently selected from hydrogen or methyl. Each R³ may be hydrogen or each R³ may be methyl. In a preferred embodiment, each R³ is methyl.

Examples of especially suitable cations are DTDTMA (ditetradecyl trimethyl ammonium), DOTMA (2,3-dioleyloxypropyl-1-trimentyl ammonium) and DHDTMA (dihexadecyl trimethyl ammonium). Cationic lipids comprising the above cations and chloride counter anions are illustrated below:

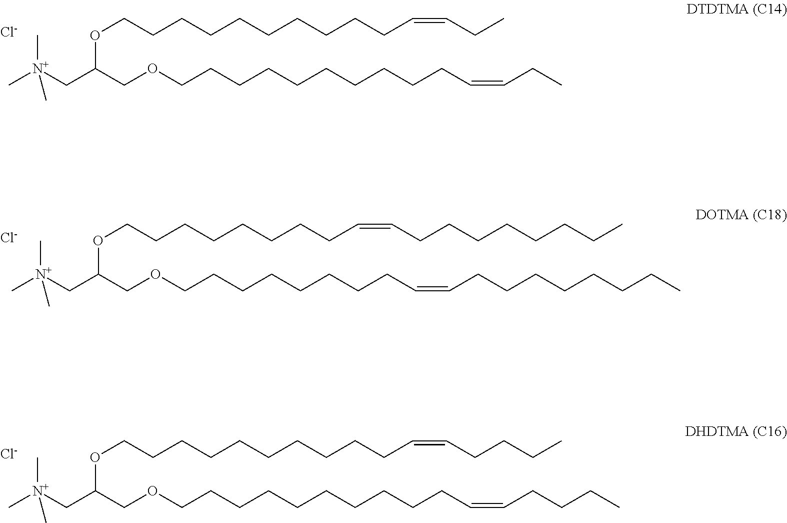

Phospholipid (b)

The term “phospholipid” refers to a lipid comprising a fatty acid chain and a phosphate group. Phospholipids are typically neutral molecules in that they do not have an overall charge, unlike a cationic lipid which is positively charged. Phospholipids are typically zwitterionic compounds comprising both positive and negatively charged components, but no overall charge. As such, phospholipids are a typically classified as neutral lipids.

Advantageously, the phospholipid is partially unsaturated. It has been found that partially unsaturated phospholipids perform better than phospholipids lacking in C═C unsaturated double bonds. Advantageously, the phospholipid adopts a conical shape. Without wishing to be bound by theory, it is thought that the conical shape of the lipid allows a complex to better fuse with the cell membrane allowing an active ingredient to be taken up into the cell as well as improve endosomal escape. The phospholipid may optionally be partially unsaturated and adopt a conical shape.

The phospholipid is advantageously of the formula (2):

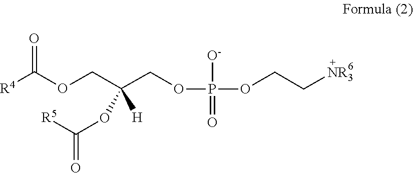

in which R⁴ and R⁵ are each independently C₁₁₋₂₃ unsaturated alkyl chain and each R⁵ group is independently selected from hydrogen and C₁₋₄ alkyl.

Advantageously, R⁴ and R⁵ are the same or different and are each independently a C₁₁₋₂₃ unsaturated alkenyl chain comprising one or two unsaturated C═C double bonds, preferably one C═C double bond. The C═C double bonds are preferably cis-. Optionally, R⁴ and R⁵ are each independently a C₁₃₋₂₁ alkenyl group, for example, a C₁₅₋₁₉ alkenyl group, such as a C₁₇ alkenyl group. R⁴ and R⁵ optionally have straight (i.e. unbranched) alkyl chains. R⁴ and R⁵ may, for example, be each independently selected from C₁₃₋₂₁, straight chain alkenyl group having one C═C unsaturated double bond; or R⁴ and R⁵ may, for example, be each independently selected from C₁₅₋₁₉, straight chain alkenyl group having one cis-C═C unsaturated double bond. R⁴ and R⁵ may, for example, each be a C₁₇, straight chain alkenyl group having one cis-C═C unsaturated double bond. Optionally, R⁴ and R⁵ are the same or different and are each independently a straight chain, unsaturated alkenyl group having one double bond. Optionally R⁴ and R⁵ are selected from —(CH₂)₆₋₁₂CH═CH(CH₂)₁₋₉CH₃, especially —(CH₂)₇₋₁₁CH═CH(CH₂)₁₋₈CH₃ or —(CH₂)₈₋₁₀CH═CH(CH₂)₁₋₇CH₃, such as —(CH₂)₁₀CH═CH(CH₂)₁CH₃, —(CH₂)CH═CH(CH₂)₇CH₃ or —(CH₂)₁₀CH═CH(CH₂)₃CH₃. Preferably, the double bond is cis and R⁴ and R⁵ are selected from —(CH₂)₆₋₁₂CH[Z]═CH(CH₂)₁₋₉CH₃, especially —(CH₂)₇₋₁₁CH[Z]═CH(CH₂)₁₋₈CH₃ or —(CH₂)₈₋₁₀[Z]CH═CH(CH₂)₁₋₇CH₃, such as —(CH₂)₇[Z]CH═CH(CH₂)₇CH₃. Preferably, R⁴ and R⁵ are the same. Each R⁶ group is independently selected from hydrogen and C₁₋₄ alkyl. Preferably, each R⁶ is independently selected from hydrogen or methyl. Each R⁶ may be hydrogen or each R⁶ may be methyl. In a preferred embodiment, each R⁶ is hydrogen.

Examples of especially suitable phospholipids are DOPE (phosphatidyl ethanolamine or 1,2-dioleoyl-sn-glycero-3-phosphoethanolamine) and DOPC (phosphatidyl choline or 1,2-dioleoyl-sn-glycero-3-phosphoethanoltrimethylamine) as illustrated below. In a preferred embodiment, the phospholipid is DOPE. DOPE is partially unsaturated and has been found to adopt a conical shape.

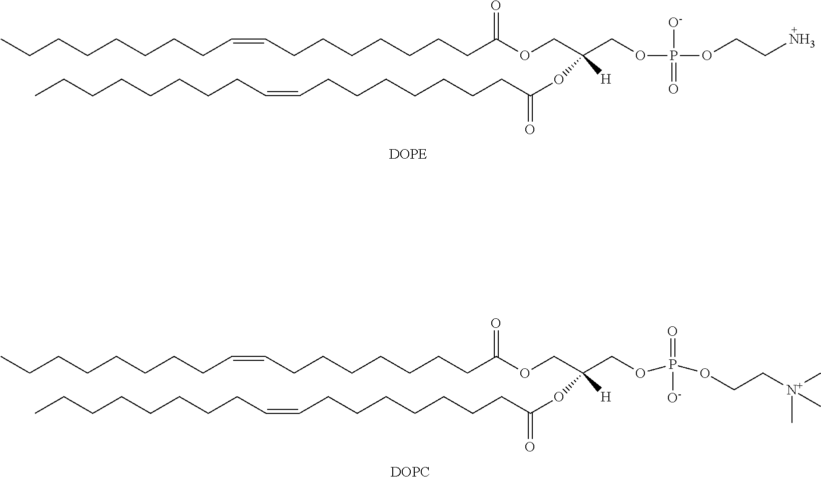 [SMILES: CCCCCCCC/C=C\CCCCCCCC(=O)OC[C@H](COP(=O)([O-])OCCN)OC(=O)CCCCCCC/C=C\CCCCCCCC ; CCCCCCCC/C=C\CCCCCCCC(=O)OC[C@H](COP(=O)([O-])OCC[N+](C)(C)C)OC(=O)CCCCCCC/C=C\CCCCCCCC ; "DOPC"]

Peptides (c)

The peptide derivative in the transfection complexes of the invention is of the formula A-B-C wherein:
A is a polycationic nucleic acid-binding component,
B is a spacer element, and
C is a cell surface receptor binding component.

The polycationic nucleic acid-binding component A is any polycation that is capable of binding to DNA or RNA. A polycation may be polycationic itself or it may have any number of cationic monomers provided the ability to bind to DNA or RNA is retained. For example, from 3 to 100 cationic monomers may be present, for example, from 10 to 20, for example from 14 to 18, for example, about 16.

The term “polycationic nucleic acid-binding component” is well known in the art and refers to polymers having at least 3 repeat cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. Such an oligopeptide may, for example, be an oligo-lysine molecule, an oligo-histidine molecule, an oligo-arginine molecule, an oligo-ornithine molecule, an oligo diaminopropionic acid molecule, or an oligo-diaminobutyric acid molecule, or a combined oligomer comprising any combination of histidine, arginine, lysine, ornithine diaminopropionic acid, and diaminobutyric acid residues. Any of the above oligopeptides may have, for example, a total of from 3 to 35, for example, from 5 to 25 residues, preferably from 10 to 20 residues, for example, from 14 to 18 residues, for example 16 residues.

An oligolysine is particularly preferred, for example, having from 3 to 35, for example, from 2 to 25, for example, form 10 to 20 lysine residues, for example, from 13 to 19, for example, from 14 to 18, for example, from 15 to 17 residues, for example, 16 residues i.e. [K]₁₆, “K” denoting lysine.

Further examples of polycationic components include dendrimers and polyethylenimine. Polyethylenimine (PEI) is a non-toxic, cross-linked cationic polymer with gene delivery potential (Proc. Natl. Acad. Sci., 1995, 92, 7297-7301). Polyethylenimine is obtainable from Fluka (800 kDa) or from Sigma (50 kDa) or alternatively pre-diluted for transfection purposes from PolyPlus-tranfection (Illkirch, France). Typically, PEI is most efficient when used in a 9 fold excess over DNA, the excess ratio being calculated as PEI nitrogen:DNA phosphate, and at pH 5 to 8. Such parameters may be optimised in a manner familiar to the person skilled in the art.

The spacer element peptide B comprises at least two amino acid residues. The spacer element peptide B is advantageously comprises three or more amino acids, for example, four or more, for example, five or more, for example, ten amino acids or more, for example up to twelve amino acids. The spacer element may, for example, comprise from 4 to 10 amino acids, such as from 6 to 8 amino acids. The amino acids may be naturally occurring or non-naturally occurring. They may have L- or D-configuration. The amino acids may be the same or different, but the use of multiple lysine residues (or other cationic amino acids suitable for use in the polycationic nucleic acid-binding component of a vector complex) should generally be avoided in the spacer as oligo-lysine sequences have activity as a polycationic nucleic acid-binding component.

The spacer element peptide B advantageously includes a cleavable portion that is susceptible to cleavage within a cell. A spacer element peptide B that includes a cleavable portion that is susceptible to cleavage within a cell may be susceptible to cleavage within the endosome, lysosome, and/or cytoplasm of a cell. Susceptible to cleavage is understood herein to mean that the element is susceptible to cleavage over a timescale during which the components A and C remain intact. Element B is cleaved more rapidly than the cellular peptide-degradation pathways take effect. The cleavable portion typically includes from 3 to 6 amino acids, for example 4 amino acids.

Preferably, the spacer element peptide is susceptible to enzymatic cleavage, reductive cleavage, or pH-dependent cleavage e.g. hydrolysis. In the case of enzymatic cleavage, in one aspect of the invention, preferred peptides are those which are susceptible to cleavage by an enzyme selected from NOX (NADH-oxidase), GILT (gamma-interferon-inducible lysosomal thiol reductase) and PDI (protein disulfide isomerase). In another aspect of the invention, preferred peptides are those which are susceptible to cleavage by an enzyme which is present in the endosome, for example an endosomal protease, such as furin or cathepsin.

Preferably the spacer element peptide B comprises a group selected from:
a) a peptide chain comprising a disulfide-linkage;
b) a peptide chain comprising an ester-linkage;
c) an amino acid sequence susceptible to cleavage by furin; and
d) an amino acid sequence susceptible to cleavage by a cathepsin enzyme

The disulfide linkage is preferably one that is stable under normal atmospheric and physiological conditions, but that may be reductively cleaved in an endosome. Similarly, an ester linkage in a peptide chain of the invention is preferably one that is stable at neutral pH, but is cleaved in the acidic environment of an endosome (for example at pH below 6.0, preferably at pH below 5.5, or at pH below 5.0).

For example, amino acid sequences susceptible to cleavage by furin include sequences selected from
i) RX¹KR; and
ii) RX²RR;

in which X¹ and X², which may be the same or different, each represents any amino acid residue (Zimmer et al., J. Biol. Chem., 2001, 276, 31642-31650; Nakayama, Biochem. J., 1997, 327, 625-635)

Preferred amino acid residues X¹ include Lys (K) and Val (V), for example Lys (K).

Preferred amino acid residues X² include Lys (K) and Val (V), for example Val (V).

For example, the cathepsin enzyme may be any suitable cathepsin enzyme (see Pillay et al., Biochem. J., 2002, 363, 417-429). For example, it may be cathepsin B. For example, amino acid sequences susceptible to cleavage by cathepsin B (see Pechar et al., Macromol. Chem. Phys., 1997, 198, 1009-1020) include sequences selected from:
iii) X³X⁴ where X³ is selected from Tyrosine (Tyr, Y), Phenylalanine (Phe, F), Leucine (Leu, L), Valine (Val, V) and Isoleucine (Ile, I) and X⁴ is selected from Glycine (Gly, G), Alanine (Ala, A) and Glutamic acid (Glu, E).

Preferably, X³ is selected from Tyrosine (Tyr, Y), Phenylalanine (Phe, F) and Leucine (Leu, L).

For example, the sequence X³X⁴ may be present as GFX³X⁴, for example as GFLG (as used in Pechar et al., Bioconjugate Chem., 2000, 11, 131-139)

As well as or instead of the cleavable portion, the spacer element peptide B may comprise a linker. The linker is preferably either a peptide, that is to say, it comprises amino acid residues, or a polyethyleneglycol group, or a mixture of the two. The amino acids may be naturally occurring or non-naturally occurring. They may have L- or D-configuration. The linker may have two or more amino acids. It may, for example, comprise three or more amino acids, for example, four or more, for example, five or more, for example, up to ten amino acids. The linker typically includes from 2 to 7 amino acids, for example from 3 to 5 amino acids. The amino acids may be the same or different. The linker may, for example, comprise the dipeptide glycine-glycine (GG) or glycine-alanine (GA). The linker may comprise a hydrophobic spacer. The linker may include the amino acid sequence XSX in which X is ε-aminocaproic acid (E-Ahx) also known as 6-aminohexanoic acid, a synthetic, i.e. non-naturally occurring, amino acid. Aminocaproic acid functions as a hydrophobic spacer. Examples of suitable linkers include GG, GA, XSXGG and XSXGA. The linker may be, for example, the dipeptide glycine-glycine (GG) or glycine-alanine (GA). Particularly preferred linkers are XSXGA and GA. Preferably, the linker is at the end of the spacer element peptide B which is bonded to the cell surface receptor binding component C.

The linker may be, or may include a polyethyleneglycol moiety. The polyethyleneglycol moiety may comprise from 1 to 30 ethylene glycol units, preferably from 1 to 15 units, more preferably from 1 to 8 units, for example from 2 to 6 units, for example 4 units.

Preferably, the linker is at the end of the spacer element peptide B which is bonded to the polycationic nucleic acid-binding component A.

The spacer may be, for example, the dipeptide glycine-glycine (GG) or glycine-alanine (GA). The spacer element peptide B may comprises a cleavable portion comprising the sequence RVRR which is bonded to the nucleic acid-binding component A and a linker which is bonded to the cell surface receptor binding component C, for example, a linker comprising or consisting of the dipeptide glycine-glycine (GG) or glycine-alanine (GA).

Preferably the cell surface receptor binding component C comprises a peptide. Where cell surface receptor binding component C comprises a peptide, the peptide may be up to 20 amino acids in length, or may be longer. The peptide generally has at least 5 amino acids but may have fewer. Generally, the peptide has any number of amino acids from 6 to 20 inclusive. Generally, it is preferred for the peptide to have 15 amino acids or fewer, more preferably 12 amino acids or fewer, most preferably 10 amino acids or fewer. Generally, it is preferred for the peptide to have 5 or more amino acids, for example, 6 or more amino acids. Most preferably, the peptide has 7 amino acids.

Preferably the cell surface receptor binding component C comprises a peptide comprising a cyclic region. Cyclic peptides may be formed by the provision of at least two cysteine residues in the peptide, thus enabling the formation of a disulphide bond. Accordingly, preferred cell surface receptor binding components C consist of or comprise a peptide having two or more cysteine residues that are capable of forming one or more disulphide bond(s). Preferably the cysteine residues flank the primary receptor binding portion.

In one embodiment of the invention, the cell surface receptor binding component C comprises an integrin-binding peptide. An integrin-binding peptide is any peptide that is capable of binding specifically to integrins found on the surface of cells. The integrin-binding peptide may be a naturally occurring integrin-binding ligand, for example, an extracellular matrix protein, a viral capsid protein, the bacterial protein invasin, a snake venom disintegrin protein, or an integrin-binding fragment of any such protein. Such integrin-binding proteins and fragments thereof may be obtained from natural sources or by recombinant techniques. It is preferable to use integrin-binding peptides, in particular because of their ease of synthesis, purification and storage, their potential for chemical modification, and their potentially low immunogenicity in vivo. Preferred integrin-binding peptides are those such as described in WO 96/15811, and especially WO 98/54347.

In this embodiment, the cell surface receptor binding component C preferably comprises a peptide selected from:
a) RGD;
b) RRETAWA;
c) LDV

In a further embodiment of the invention, the cell surface receptor binding component C comprises a peptide which is capable of binding to human airway epithelial (HAE) cells. Preferred HAE cell-binding peptides are those such as described in WO 02/072616. In this embodiment, the cell surface receptor binding component C preferably comprises a peptide selected from
a) X⁵SM;
b) LX⁶HK;
c) PSGX⁷ARA;
d) SX⁸RSMNF; and
e) LX⁹HKSMP;

in which X⁵ is a basic amino acid residue, X⁶ is Q or P, X⁷ is A or T, X⁸ is an acidic amino acid residue and X⁹ is P or Q.

Preferably, the cell surface receptor binding component C comprises a peptide selected from
a) X⁵SM;
b) LX⁶HK; and
c) PSGAARA,

in which X⁵ is a basic amino acid residue and X⁶ is Q or P.

Preferably X⁵ is K or R. Preferably X⁶ is P. Preferably X⁷ is A. Preferably X⁸ is E or Q. More preferably X⁸ is E. Preferably X⁹ is P. Accordingly, preferred peptides are those comprising a sequence selected from LQHKSMP, LPHKSMP, VKSMVTH, SERSMNF, VGLPHKF, YGLPHKF, PSGAARA, SQRSMNF and PSGTARA.

In another embodiment of the invention, the cell surface receptor binding component C comprises a peptide which is capable of binding to human dendritic cells.

Preferred human dendritic cell-binding peptides are those such as described in WO 2004/108938. For example, such a peptide may be selected from a peptide comprising an amino acid sequence selected from:
a) PX¹⁰X¹¹X¹²T;
b) PSX¹³S;
c) QX¹⁴X¹⁵X¹⁶Q;
d) SX¹⁷S,

in which X¹⁰, X¹¹ and X¹², which may be the same or different, each represents an amino acid residue;

X¹³ represents an amino acid residue;

X¹⁴ and X⁶, which may be the same or different, each represents an amino acid residue, and

X¹⁵ represents an amino acid residue having an amide side chain, for example, N or Q.

X¹ represents an amino acid residue having an aliphatic side chain, for example, L or I.

In a preferred embodiment, the cell surface receptor binding component C comprises a peptide selected from:
a) CRGDCLG;
b) CRGDCLG;
c) ACDCRGDCFCG;
d) CRGDMFGCA;
e) CRRETAWACG;
f) CRGEMFGCA;
g) CSERSMNFCG;
h) CYGLPHKFCG; and
i) CLPHKSMPCG.

In one embodiment, the receptor binding component C optionally comprises the amino acid sequence LX⁶HK in which X⁶ is Q or P. Preferably X⁶ is P. The cell surface receptor binding component C preferably comprises the amino acid sequence YGLPHKF. The cell surface receptor binding component C advantageously comprises a peptide comprising a cyclic region. Cyclic peptides may be formed by the provision of at least two cysteine residues in the peptide, thus enabling the formation of a disulphide bond. Accordingly, preferred cell surface receptor binding components C consist of or comprise a peptide having two or more cysteine residues that are capable of forming one or more disulphide bond(s). Preferably the cysteine residues flank the primary receptor binding portion. The cell surface receptor binding component C optionally comprises the amino acid sequence CYGLPHKFCG.

The peptide of structure A-B-C may for example comprise a nucleic acid binding polycation, such as polylysine, bonded to a spacer element B, bonded to the receptor binding component.

The spacer element optionally comprises a linker portion comprising the amino acid sequence XSXGA or GA. The spacer element is optionally bonded to a cell surface receptor binding component YGLPHKF, optionally flanked by two cysteine residues.

The peptide may comprise as polylysine, a spacer element, e.g. comprising the amino acid sequence XSXGA or GA, and a cell surface receptor binding component YGLPHKF, optionally flanked by two cysteine residues.

Ribonucleoprotein (RNP) (d)

The CRISPR-associated protein present in the RNP is an RNA guided protein useful in CRISPR/Cas9 systems. The CRISPR-associated protein may be a CRISPR-associated nuclease endonuclease protein, such as Cas9, or a homologue thereof. The CRISPR-associated protein can have a function other than as a nuclease that produces a double stranded break in the DNA of the target genome, for example, the CRISPR-associated protein may be an engineered protein that only nicks (cuts) one strand of DNA, or a disabled nuclease protein that does not make any breaks but carries other enzymatic functions to the targeted region enabling enzymatic modifications such as methylation. The CRISPR-associated protein may be a fusion protein able to fuse a fragment of DNA into a genome without a cutting function. CRISPR-associated protein typically comprises from 800 to 2000 amino acid residues, such as from 900 to 1800 residues, especially from 1000 to 1700 residues.

The guide RNA is advantageously a single guide RNA (sgRNA) comprising a single fragment of RNA. An sgRNA molecule typically comprises a portion configured to bind to the protospacer in the DNA target and a portion that binds to the CRISPR-associated protein. The guide RNA may alternative comprise more than one RNA fragments, for example comprising a combination of a CRISPR-RNA (crRNA) molecule that is configured to bind to the protospacer in the DNA target and a trans-activating crRNA (tracrRNA) molecule that binds to the CRISPR-associated protein.

In addition to the CRISPR-associated protein and guide RNA, the ribonucleoprotein (RNP) complex may optionally include a DNA repair template. The DNA repair template guides the cellular repair process allowing insertion of a specific DNA sequence. The DNA repair template may include a homologous donor DNA molecule. Systems in which the RPN includes a DNA repair template may be used to knock in replacement DNA into a genome of a cell.

Ribonucleoprotein (RNP) Delivery System

The ribonucleoprotein (RNP) delivery system of the invention comprises:
(a) a cationic lipid;
(b) a phospholipid;
(c) a peptide of the structure A-B-C in which:
   A is a polycationic nucleic acid-binding component,
   B is a spacer element comprising two or more amino acid residues, and
   C is a cell surface receptor binding component; and
(d) a ribonucleoprotein (RNP) complex comprising a CRISPR-associated protein and guide RNA.

The invention further provides a liposome comprising (a) a cationic lipid, (b) a phospholipid and (c) a peptide as defined above. It has been found that the combination of (a) a cationic lipid, (b) a phospholipid and (c) a peptide as defined above provides a liposome that when combined with and RNP complex provides is a particularly effective transfection complex for the delivery of an RNP complex to cells both in vitro and in vivo.

The RNP delivery system is typically in the form of a nanoparticles, also referred to as a “nanocomplex”. The nanoparticles typically self-assemble, for example on mixing a preassembled RNP complex with the lipid and peptide components in a suitable liquid vehicle. The nanoparticles of the invention typically have a Z-average particle size (D_{z}) of from 50 to 300 nm, for example from 80 to 200 nm, such as from 85 to 160 nm. Advantageously, the nanoparticles have a Z-average particle size (D_{z}) of less than 150 nm, for example, less than 130 nm, such as less than 125 nm, especially less than 120 nm, e.g. a Z-average particle size (D_{z}) of from 90 to 120 nm. The Z-average (D_{z}) is the intensity based harmonic mean as described by the equation: D_{z}=ΣSᵢ/Σ(Sᵢ/Dᵢ) where Sᵢ is the scattered intensity from particle i and Dᵢ is the diameter of particle i, e.g. as measured using on a Malvern Nano ZS Zetasizer. Advantageously, all the essential components of the RNP delivery system of the invention are bound together in a single complex, e.g. in the form of a self-contained nanoparticle. Advantageously, no external enhancer elements or promoter compounds are needed other than the components bound into the nanoparticles.

Particularly effective combinations include a combination of (a) a cationic lipid comprising a cation the formula (1) in which R¹ and R² the same or different and are selected from —(CH₂)₇₋₁₁CH[Z]═CH(CH₂)₁₋₈CH₃ or —(CH₂)₈₋₁₀[Z]CH═CH(CH₂)₁₋₇CH₃, such as —(CH₂)₁₀[Z]CH═CH(CH₂)₁CH₃, —(CH₂)₈[Z]CH═CH(CH₂)₇CH₃ or —(CH₂)₁₀[Z]CH═CH(CH₂)₃CH₃ and R³ is methyl or hydrogen; (b) a phospholipid of the formula (2) in which —(CH₂)₇₋₁₁CH[Z]═CH(CH₂)₁₋₈CH₃ or —(CH₂)₈₋₁₀[Z]CH═CH(CH₂)₁₋₇CH₃, such as —(CH₂)₇[Z]CH═CH(CH₂)₇CH₃ and R⁵ is methyl or hydrogen; and (c) a peptide as defined above comprising the sequence YGLPHKF or SERSMNF, especially YGLPHKF. For example, the liposome may comprise a combination of (a) DTDTMA, DHDTMA or DOTMA; with (b) DOPE or DOPC, especially DHDTMA with DOPE; and (c) peptide comprising a nucleic acid binding polycation A, such as polylysine, bonded to a spacer element B bonded to a cell surface receptor binding component YGLPHKF or SERSMNF, especially YGLPHKF, optionally flanked by two cysteine residues, especially XSXGA or GA bonded to CYGLPHKFCG. For example, the liposome may comprise a combination of DHDTMA (C16) or DOTMA (C18) with DOPE and XSXGA or GA bonded to CYGLPHKFCG or CSERSMNFGC, especially of DHDTMA (C16) with DOPE and XSXGA or GA bonded to CYGLPHKFCG.

In particularly preferred embodiments, the liposome of the RNP delivery system comprises (i) DTDTMA, DOPE and K₁₆-B-YGLPHKF; (ii) DOTMA, DOPE and K₁₆-B-YGLPHKF; or (iii) DHDTMA, DOPE and K₁₆-B-YGLPHKF, where B is a linker peptide of from 2 to 10 amino acid residues. In one embodiment, the liposome comprises (i) DTDTMA, DOPE and K₁₆-GA-CYGLPHKFCG; (ii) DOTMA, DOPE and K₁₆-GA-CYGLPHKFCG; or (iii) DHDTMA, DOPE and K₁₆-GA-CYGLPHKFCG. In an especially preferred embodiment, the liposome comprises (i) DHDTMA (C16), DOPE and K₁₆-GA-CYGLPHKFCG.

It has been found that the ratio of components in the genome editing system effects the efficacy of transfection. In particular, the ratio of total lipids (i.e. the total of (a)+(b) cationic, phospholipids and other optional lipids present in the genome editing system) to (c) peptide components in the system has been found to effect transfection efficacy. The ratio of (a)+(b) total lipids to (c) peptide is optionally in the range of from about 1:4 to about 1:1 parts by weight, for example, from about 1.5:4 to about 4:3.8 parts by weight, especially from 1.8:4 to 4:3.5, such as from 2.6:4 to 3.4:4 parts by weight or 2.8:4 to 3.2:4 parts by weight. It has been found that optimal transfection efficacy is obtained with a ratio of (a)+(b) total lipids to (c) peptide of about 1:2 to about 3:4, especially about 3:4 parts by weight.

The ratio of components (d) RNP (including CRIPSRP-associated protein, guide RNA and any optional DNA repair template) to (c) peptide is advantageously about 1:4 parts by weight. Advantageously the ration of components (d):(c) is from about 1:8 to about 1:2 parts by weight, especially from about 1:6 to about 1:3 parts by weight, such as from 1:5 to 1:4.5 parts by weight. It has been found that optimal transfection efficacy is obtained with a ratio of (d) RNP (a)+(b) total lipids to (c) peptide of about 1:2 to about 3:4, especially about 3:4 parts by weight.

Medical Uses

It has been found that the ribonucleoprotein (RNP) delivery system of the invention improves the targeting of an RNP-containing vector complex, e.g. to tumour cells. In particular, the RNP delivery system has been found to allow for targeted, specific editing or correction of specific mutations in cells with the potential to disrupt a gene that may be having a deleterious effect, or correcting a gene mutation such as in recessive genetic conditions, such as cystic fibrosis or Duchenne muscular dystrophy, to restore expression.

The invention provides the use of the RNP delivery system of the invention in transfection. The genome editing system is advantageously useful in editing the genome found in specific cells. Genome editing may involve removal of DNA from a genome and, optionally, knocking in replacement DNA.

The RNP delivery system of the invention may be used to remove sequences of up to 100 genes from a genome, typically removing from about 5 to 30 genes. When the RNP complex includes an optional DNA repair template the genome editing system may be used to insert a specific DNA sequence into the genome.

The RNP delivery system and liposome of the invention may find use in therapy, for example, the treatment of cancer. The invention, thus provides a method of treating cancer comprising administering an RNP delivery system of the first aspect of the invention to the patient in an effective amount.

The RNP delivery system of the invention may be administered in a pharmaceutical composition of the second aspect of the invention, which comprises the RNP delivery system in admixture or conjunction with a pharmaceutically suitable carrier.

The invention further provides an RNP delivery system of the first aspect of the invention for use as a medicament or a vaccine.

The optional DNA repair template may be any suitable nucleic acid. It may be DNA or RNA or a chemically modified nucleic acid mimetic, for example a PNA molecule. It may, for example, code for a protein that has a utility in the target cell. Advantageously, the DNA repair template is cellular messenger RNA (mRNA).

The invention also provides processes for the production of an RNP delivery system of the first aspect of the invention. In particular, the invention provides a method of making an RNP delivery system as described herein, the method comprising the steps of:

(i) providing a ribonucleoprotein (RNP) complex comprising a CRISPR-associated protein and guide RNA;

(ii) combining the ribonucleoprotein complex from step (i) with lipid component comprising a phospholipid and a cationic lipid; and

(iii) adding a peptide containing (A) a polycationic nucleic acid-binding component, (B) a spacer element comprising two or more amino acid residues and (C) a cell surface receptor binding component to the mixture from step (ii) to form the RNP delivery system.

The RNP delivery system of the invention may be used in the editing of a gene in mammalian cell. The gene may be edited by removing a defect in the gene, i.e. a defective portion of DNA, and/or knocking in (insertion) of replacement DNA. The invention provides a method of editing of a gene in mammalian cell comprising transfecting the cell with the RNP delivery system of the firs aspect of the invention. The method may be performed ex vivo or in vivo. Unlike known non-viral lipid based transfection systems the RNP delivery system of the invention has been found to be effective in the in vivo transfection of cells. The RNP delivery system of the invention may be used in the editing of a gene in mammalian cell to correct a defect and/or a deficiency in a gene. The invention provides a method for the treatment or prophylaxis of a condition caused in a human or in a non-human animal by a defect and/or a deficiency in a gene which comprises administering an RNP delivery system of the first aspect of the invention the human or to the non-human animal. The term “a defect and/or a deficiency in a gene” as used herein denotes not only a defect or deficiency in the coding region of a gene, but a defect or deficiency in a control element for the gene, for example, a control element in trans or in cis, or a defect or deficiency in any other element that is involved in the transcription or translation of the gene, whether directly or indirectly.

### EXAMPLES

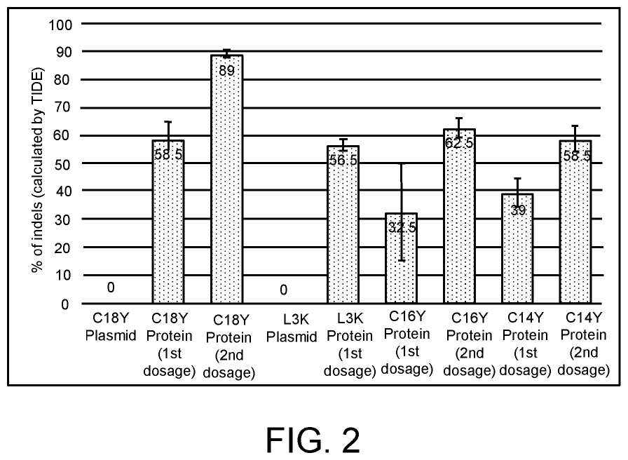

In the following examples the receptor-targeted nanocomplexes (RTN) (i.e. the liposome part of the RNP delivery system other than the RNP complex or nucleic acid “payload”) are denoted using the abbreviation “Cnn DXXX P”, where Cnn is the cationic lipid, e.g. C14 (DTDTMA), C18 (DOTMA) or C16 (DHDTMA); DXXX is the phospholipid, e.g. DOPE (phosphatidyl ethanolamine or 1,2-dioleoyl-sn-glycero-3-phosphoethanolamine), DOPC (phosphatidyl choline or 1,2-dioleoyl-sn-glycero-3-phosphoethanoltrimethylamine) or DSPC (1,2-distearoyl-sn-glycero-3-phosphocholine); and P is the peptide, e.g. Peptide Y (Y) or Peptide E (E). Where receptor-targeted nanocomplexes (RTN) are denoted using the abbreviation “CnnP” (e.g. in FIG. 2), Cnn is the cationic lipid, P is the peptide and the phospholipid is DOPE. The RNP delivery systems of the invention may be referred to in the examples as “nanocomplexes”.

Materials

The peptides described below were synthesized using standard instruments and techniques.

| Peptide | Sequence | Mass (g.mol⁻¹) |
|---|---|---|
| K16 | KKKKKKKKKKKKKKKK | 2068 |
| Y | (K)₁₆GACYGLPHKFCG | 3303.28 |
| E | (K)₁₆GACSERSMNFCG | |

The oligolysine-peptides may be synthesised using standard solid phase oligopeptide synthesis methods, including the method described below:

Linear peptide sequences: Peptides (e.g. Peptides Y and E) may be synthesized on a SYRO automated peptide synthesizer on a 20 μmol scale using 2 ml syringes with Teflon frits and 500 μl coupling volume. Fmoc-Gly preloaded NovaSyn TGT resin or Fmoc-Gly-2-Cl-Trt-resin were used for these sequences. Fmoc-Peg4-COOH was synthesized following a procedure reported previously (see synthesis of Fmoc-Haa4-COOH at page 82 of WO 2005/117985-Fmoc-Haa4-COOH was the name given to Fmoc-Peg4-COOH in that specification). The TGT resin was initially swelled for 10 min, however the 2-Cl-Trt resin needed a prolonged initial swelling time (some hours) in DMF. Routine coupling was performed with HBTU (in DMF) and DIPEA (in NMP) using a fourfold excess of reagents. Fmoc was cleaved with a 40% solution of piperidine in DMF for 3 min and a 20% solution for 10 min. Synthesis cycles consisted of 40 min coupling time, 3 min for Fmoc deprotection with 40% piperidine, another 10 min for Fmoc deprotection with 20% piperidine and washing steps. After synthesis and the last wash cycle with DMF, peptides were washed with DCM, methanol and diethyl ether (3 times each) using the “manual”/“empty” function of the Syro. Suction was applied for some more time to help evaporate the ether.

On-resin disulfide bondformation: To form disulphide bridges on resin, the resin was placed in a syringe with PE frit and swelled in DMF. After removal of excess DMF a freshly prepared solution of iodine in a minimum amount of DMF (e.g. 500 μl for a 2 ml syringe, 10 eq iodine to resin loading) was added and the syringe was vortexed during 4 h for 20 s every 4 min. The reagent solution was removed and the resin was washed 10 to 20 times with DMF, and 3 times each with DCM, methanol and ether.

Cleavage and deprotection: The syringes were transferred to the fume hood for cleavage. Cleavage was performed with a cocktail of 95% TFA, 2.5% TIS and 2.5% H₂O. A minimum amount of freshly prepared cocktail was added to cover the resin (e.g. <500 μl in the 2 ml syringes). After 4 h the cleavage solutions were passed into polypropylene (PP) tubes using a plunger and the resins were washed with another small amount of cleavage cocktail (e.g. 200 μl in the 2 ml syringes). Then the peptides were precipitated with ether (e.g. to the combined fractions of the 2 ml syringes some 4 ml of diethyl ether were added). The PP tubes were kept in the freezer for at least 15 min, then centrifuged at 3000 rpm for 3 min and solution was decanted from the peptide pellet. Centrifugation and decantation were repeated twice with about 2 ml of ether. Finally the peptides were dissolved in water or tBuOH/water (4:1) and freeze-dried. Some peptide sequences showed very poor solubility and sometimes several lyophilisation/dissolution processes with different solvent mixtures (water, tBuOH or acetonitrile) were necessary to obtain a fluffy peptide.

The peptide was analyzed by reverse phase HPLC and purified by reverse phase HPLC to >90% pure. Mass spectra were recorded using the Micromass Quattro ES-MS (Software: Masslynx) and the masses are recorded in the table IB.

Peptide Y was purchased from AMS Bio Ltd., Birmingham, UK, and synthesised using semi-automated peptide synthesis chemistry. The peptide was analysed by reverse phase HPLC and purified where necessary by reverse phase HPLC to 85% pure.

K16 was purchased as described previously (Hart et al., Lipid-mediated enhancement of transfection by a nonviral integrin-targeting vector. Hum Gene Ther., 1998, 9, 575-585).

All these freeze-dried peptides were diluted at 10 mg/ml in water and stored at −20° C. during several months. Once thawing, aliquots of peptides are kept at 4° C. during several weeks.
DTDTMA (C14), DOTMA (C18) and DHDTMA (C16) were prepared according to the method described in Hurley C A, Wong J B, Hailes H C and Tabor A B, Assymetric Synthesis of Dialkyloxy-3-alkylammonium Cationic Lipids. J. Org. Chem. 2004, 69:980-983.
DOPE is available from Avanti Polar Lipids, Alabaster, Ala., USA.
DSPC is available from Avanti Polar Lipids, Alabaster, Ala., USA.
DOPC is available from Avanti Polar Lipids, Alabaster, Ala., USA.
Alt-R® S.p. Cas9 nuclease is available from Integrated DNA Technologies (IDT).
Alt-R® gRNA guide RNA is available from Integrated DNA Technologies (IDT).
NHBE BMI-1 GFP cells are normal human bronchial epithelial (NHBE) cells in which green fluorescent protein (GFP) is expressed under the endogenous transcriptional regulatory elements of the Bmi-1 gene, which are available from Lonza.
CFBE BMI-1 cells are Cystic fibrosis bronchial epithelial cells from Epithelix (Epithelix SàRL, Genève, Switzerland), transduced by BMI.
Lipofectamine 3000 (L3K) is available from Thermo Fischer Scientific.
Opti-MEM (Life Technologies) reduced serum media is available from Thermo Fischer Scientific.
BEGM is bronchial epithelial growth medium.
Lipofectamine™ CRSPRMAX™ Cas9 Transfection Reagent (Irvtrogen) is available from Thermo Fischer Scientific.
Nuclease-Free Duplex Buffer is available from Integrated DNA Technologies (IDT).

### Example 1—Transfection

Normal human bronchial epithelial (NHBE) cells in which green fluorescent protein (GFP) is expressed under the endogenous transcriptional regulatory elements of the Bmi-1 gene (NHBE BMI-1 GFP cells) (Lonza) were seeded in 24 well plates at a density of 0.6×105 cells per well in a total volume of 1 ml bronchial epithelial growth medium (BEGM) 24 hours before transfection.

Alt-R® S.p. Cas9 nuclease (IDT), and Alt-R® gRNA (IDT) were mixed at a 4:1 weight ratio and incubated for 5 m at room temperature, to allow self-assembly. Two concentrations of RNP were compared: 500 ng Cas9+125 ng gRNA and 1000 ng Cas9+250 ng gRNA.

Nanocomplexes (i.e. RNP delivery systems of the invention) were prepared in 100 μl reduced serum medium (Opti-MEM) per well at a weight ratio of 1:3:4 RNP:total lipid (L):peptide (P). Components were incubated for 30 minutes at room temperature, allowing the complexes to self-assemble. CRISPRMAX transfections were assembled as per manufacturer's instructions. Nanocomplexes were then diluted in 1 ml Opti-MEM per well and mixed by vigorous pipetting.

BEGM media was removed from cells and diluted nanocomplexes added. Cells were incubated at 37° C. for 4 hours, after which Opti-MEM was replaced with BEGM. Cells were maintained for 8 days before assessment by Flow Cytometry. GFP fluorescence was measured to assess GFP knockdown.

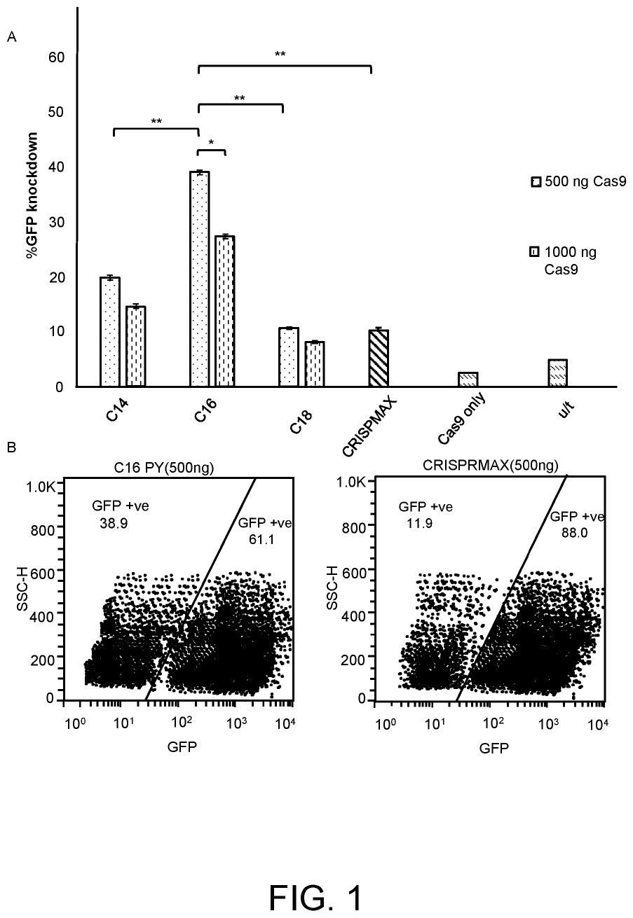

FIG. 1 (A) shows the GFP knockdown (%) in NHBE BMI-1 GFP cells following transfection with Cas9 RNP complex using either DTDTMA (C14), DHDTMA (C16) or DOTMA (C18)/DOPE liposomes and peptide Y, at a weight ratio of 1:3:4 RNP:L:P. Two concentrations of RNP were used: 500 ng Cas9 protein with 125 ng gRNA (purple) and 1000 ng Cas9 protein with 250 ng gRNA (pink). For CRISPRMAX, 500 ng Cas9 protein with 125 ng gRNA was used, as per manufacturer's instructions. GFP fluorescence was measured by Flow Cytometry 8 days after transfection.

FIG. 1 (B) shows representative density plots of GFP knockdown with C16 DOPE PY and CRISPRMAX (500 ng Cas9 protein) N=2 in all experiments, error bars=SEM. Student's T.Test*p≤0.05**p≤0.01.

It was observed that using a lower concentration of RNP resulted in a higher transfection efficiency (p=0.0137 using DHDTMA (C16)/DOPE). DHDTMA (C16)/DOPE significantly out-performed both DTDTMA (C14) and DOTMA (C18)/DOPE (p=0.0099 and p=0.0123), with a 2-fold and 4-fold increase in GFP knockdown respectively. Notably, delivery of 500 ng Cas9 protein and 125 ng gRNA was significantly improved using DHDTMA (C16)/DOPE and peptide Y, when compared to the same concentration being delivered by CRISPRMAXT^{M}, a reagent specifically optimised for protein delivery, resulting in a 4-fold increase in GFP knockdown (p=0.0086) (FIG. 1).

### Example 2—Characterisation of Nanoparticles Using Dynamic Light Scattering

Nanocomplexes were prepared in 100 μl water per well at a weight ratio 1:3:4 RNP:total lipid (L):peptide (P). Components were incubated for 30 minutes at room temperature, allowing the complexes to self-assemble. Nanocomplexes (containing 2 μg RNP complex) were then diluted in 1 ml water per well and mixed by vigorous pipetting.

Size and charge of RNP complexes were determined by photocorrelation spectroscopy (PCS) (also known as dynamic light scattering; DLS) measurements on a Malvern Nano ZS Zetasizer (Malvern Instruments, England) (Table 1).

TABLE 1
Composition and associated hydrodynamic size and zeta potential
of nanocomplexes as measured by dynamic light scattering.

| | | Z-Average (nm) | | PDI | | Zeta potential (mV) | |
|---|---|---|---|---|---|---|---|
| Nanoformulation | | Average | St. Dev | Average | St. Dev | Average | St. Dev |
| mRNA | C18 COPE Y Cas9 mRNA | 104.4 | 1.0 | 0.230 | 0.0 | 48.1 | 3.6 |
| Protein | C14 DOPE Y Cas9 RNP | 142.6 | 3.0 | 0.437 | 0.0 | 61.0 | 5.9 |
| | C16 DOPE Y Cas9 RNP | 95.7 | 0.7 | 0.211 | 0.0 | 35.5 | 2.3 |
| | C18 DOPE Y Cas9 RNP | 90.0 | 0.5 | 0.180 | 0.0 | 60.4 | 4.7 |

Cas9 mRNA+gRNA was compared to Cas9 RNP complexes. Nanocomplexes were formulated with either C14, C16 or C18/DOPE and Peptide Y, at a weight ratio of 1:3:4. 500 ng Cas9 protein was formulated with 125 ng gRNA. 300 ng Cas9 mRNA was formulated with 200 ng gRNA. Nanoparticle size decreased in the trend C18<C16<C14 while the polydispersity index (PDI), a measure of size variation, also decreased with the same trend.

### Example 3—Characterisation of Nanoparticles Using Dynamic Light Scattering

Nanocomplexes were prepared in 100 ul water per well at a weight ratio of 1:1:4, 1:2:4, 1:3:4 and 1:4:4 ribonucleoprotein complex (RNP):total lipid (L):peptide (P). Components were incubated for 30 minutes at room temperature, allowing the complexes to self-assemble. Nanocomplexes (containing 2 μg RNP complex) were then diluted in 1 ml water per well and mixed by vigorous pipetting.

Size and charge of RNP complexes were determined by photocorrelation spectroscopy (PCS) (also known as dynamic light scattering; DLS) measurements on a Malvern Nano ZS Zetasizer (Malvern Instruments, England) (Table 2).

TABLE 2
Composition and associated hydrodynamic size and zeta potential of
Cas9 RNP nanocomplexes as measured by dynamic light scattering.

| Nano- | Z-Average (nm) | | PDI | | Zeta potential (mV) | |
|---|---|---|---|---|---|---|
| formulation | Average | St. Dev | Average | St. Dev | Average | St. Dev |
| 1:4 RNP:P | 196.3 | 0.9 | 0.365 | 0.0 | 24.567 | 1.3 |
| 1:4 RNP:L | 183.2 | 5.4 | 0.397 | 0.0 | 40.500 | 1.5 |
| 1:1:4 RNP:L:P | 177.4 | 7.0 | 0.263 | 0.0 | 44.320 | 1.8 |
| 1:2:4 RNP:L:P | 111.0 | 2.8 | 0.227 | 0.0 | 33.188 | 4.0 |
| 1:3:4 RNP:L:P | 108.5 | 0.9 | 0.221 | 0.0 | 42.100 | 3.0 |
| 1:4:4 RNP:L:P | 165.8 | 5.3 | 0.400 | 0.0 | 46.800 | 1.0 |

Nanocomplexes contained 500 ng Cas9 protein and 125 ng gRNA, and were formulated using C18 and peptide Y, at various weight ratios. The optimal ratio of RNP:total lipid (L):peptide (P):was 1:3:4 with the smallest size and lowest PDI value (Table 2). Peptide alone, or lipid alone formed larger, more variable particles than the RLP mixture with high polydispersity index (PDI) values.

### Example 4—Transfection

Primary Cystic fibrosis bronchial epithelial (CFBE) cells (CFBE BMI-1) were seeded on a 24 well collage coated plate at a density of 0.05×10⁶ cells per well.

A targeting gRNA was purchased from Integrated DNA Technologies (IDT) as two systems (crRNA and tracrRNA) as lyophilized vials. The gRNA was re-suspended in Nuclease-Free Duplex Buffer (IDT) to make 100 μM stock, then diluted with Nuclease-Free Duplex Buffer to a final equimolar duplex concentration (5 μM). The solution was denatured at 95° C. for 5 minutes then left at room temperature for 20 minutes to anneal.

To form the ribonucleoprotein (RNP) complex, Alt-R® S.p Cas9 (IDT) protein was mixed with the 5 μM crRNA:tracrRNA guide RNA solution in an equal molar amount in Opti-MEM (Life Technologies) and incubated for 5 minutes to assemble.

RNP delivery systems of the invention as receptor-targeted nanocomplexes (RTN) were formulated by mixing lipid (C14, C16 or C18 in a 1:1 ratio with DOPE) with the ribonucleoprotein, then peptide Y was added finally. The weight ratio for the RNP delivery systems is 1:3:4 (RNP:Lipid:peptide). The RTN was prepared in Opti-MEM then incubated for 30 mins at room temperature to allow the RTNs to form.

As a comparison, the transfection reagent Lipofectamine® 3000 (Life Technologies) was prepared with the RNP complex at a weight ratio of 3:1 (reagent:RNP) or with plasmid DNA (1 ag) expressing both Cas9 nuclease and single guide RNA at a weight ratio of 3:1 (reagent DNA) in Opti-MEM, then incubated for 10 minutes at room temperature.

Also as a comparison, receptor-targeted nanocomplexes (RTN) were formulated by mixing lipid (C14, C16 or C18 in a 1:1 ratio with DOPE) with plasmid DNA (1 μg) encoding both gRNA and Cas9, then peptide Y was added finally. The RTN was prepared in Opti-MEM then incubated for 30 minutes at room temperature to allow the RTNs to form.

After the incubation time (30 minutes for the RTN, RNP delivery system of the invention and 10 min for Lipofectamine® 3000) the complete media was removed then the transfection complexes added to the cells in the plate. The primary CFBE cells were transfected either with 1 μg of plasmid DNA encoding both Cas9 and gRNA or with RNP comprising 500 μg of cas9 (nuclease protein) and 125 ng of gRNA. Then, the plate was centrifuged for 5 minutes at 200 relative centrifugal force to enhance the complex uptake, followed by incubation at 37° C. for 4 hours. After that, the Opti-MEM was removed by aspiration and replaced with BEGM.

The genomic DNA was harvested from the cells after 48 hours followed by amplification by a PCR reaction for the targeting region. To determine the indel %, Sanger sequencing performed from un-transfected and transfected cell pool, then the chromatogram sequence was analysed using the TIDE web tool that uses an algorithm to reconstruct the spectrum of indels from the sequence traces, the sequence being uploaded to http://tide.nki.nl/.

FIG. 2 shows the Indels analysis of targeting CFTR by the RNP delivery system of the invention, compared with plasmid RNA delivery using receptor-targeted nanocomplexes, RNP delivery using Lipofectamine 3000 (L3K), and plasmid RNA delivery using Lipofectamine 3000 (L3K). The analysis shows the efficiency of indels assessed by TIDE (Tracking of Indels by Decomposition) algorithmic analysis to compare the sequence of transfected and control samples. The results are represented as the mean±SD (standard deviation).

The primary CFTR cells are in general difficult to transfect. The indels were undetectable after transfection with plasmid encoding both gRNA and Cas9 formulated either with Lipofectamine 3000 (L3K plasmid) or RTN (C18 DOPE Y plasmid) (FIG. 2). Surprisingly, the cells edited with the RNP delivery system of the invention (C18 DOPE Y protein, C16 DOPE Y protein and C14 DOPE Y protein all showed substantial levels of transfection. The TIDE assay analysis shows the highest indels when the RNP formulated with C18/DOPE and peptide Y (58.5%) which it was as efficient as the commercial lipofection reagent Lipofectamine 3000 (L3K). Furthermore, an accumulation effect has been demonstrated by repeat transection dosing with each lipid system, and for lipid systems comprising DOTMA (C18) and DOPE resulted in which almost all cells being edited (89%) after a second dosage.

1. A ribonucleoprotein (RNP) delivery system comprising:
(a) a cationic lipid;
(b) a phospholipid;
(c) a peptide of the structure A-B-C in which:
A is a polycationic nucleic acid-binding component,
B is a spacer element comprising two or more amino acid residues, and
C is a cell surface receptor binding component; and
(d) a ribonucleoprotein (RNP) complex comprising a CRISPR-associated protein and guide RNA.

2. The RNP delivery system of claim 1, wherein C, the cell surface receptor binding component of the peptide (c), comprises an amino acid sequence selected from:
(i)[[a)]]
RGD;
(ii)[[b)]]
(SEQ ID NO: 8)
RRETAWA;
(iii)[[c)]]
LDV;
(iv)[[d)]]
X⁵SM;
(v)[[e)]]
(SEQ ID NO: 9)
LX⁶HK
(vi)[[f]];
(SEQ ID NO: 10)
PSGX⁷ARA;
(vii)[[g)]]
(SEQ ID NO: 11)
SX⁸RSMNF;
and
(viii)[[h)]]
(SEQ ID NO: 12)
LX⁹HKSMP;
in which X⁵ is a basic amino acid residue, X⁶ is Q or P, X⁷ is A or T, X⁸ is E or another acidic amino acid residue and X⁹ is P or Q.

3. The RNP delivery system of claim 1, wherein the phospholipid (b) is of the formula (2): 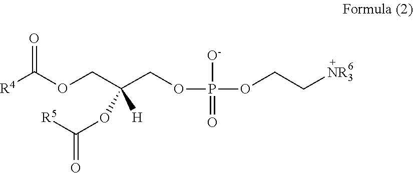
in which R⁴ and R⁵ are each independently C₁₁₋₂₃ unsaturated alkyl chain and each R⁶ group is independently selected from hydrogen and C₁₋₄ alkyl.

4. The RNP delivery system of claim 1, wherein cationic lipid (a) is selected from DTDTMA (ditetradecyl trimethyl ammonium), DOTMA (2,3-dioleyloxypropyl-1-trimentyl ammonium) and DHDTMA (dihexadecyl trimethyl ammonium).

5. The RNP delivery system of claim 1, wherein the cell surface receptor binding component comprises the amino acid sequence YGLPHKF (SEQ ID NO: 19), CYGLPHKFCG (SEQ ID NO: 33), SERSMNF (SEQ ID NO: 17) or CSERSMNFCG (SEQ ID NO: 32).

6. The RNP delivery system of claim 1, wherein A the polycationic nucleic acid-binding component of the peptide (c) comprises or consists of from 5 to 25 histidine, arginine, lysine, ornithine diaminopropionic acid, and diaminobutyric acid residues, and especially consists of from 14 to 18 lysine residues.

7. The RNP delivery system of claim 1, having:
from 0.6 to 2.0 parts by weight RNP complex (d),
from 2.6 to 3.4 parts by weight total lipids (a)+(b), and
from 3.5 to 5.0 parts by weight peptide (c).

8. The RNP delivery system of claim 1, wherein the CRISPR-associated protein is an endonuclease having from 900 to 1800 residues.

9. The RNP delivery system of claim 1, wherein the guide RNA is (i) a single guide RNA (sgRNA) comprising a portion configured to bind to the protospacer in the DNA target and a portion that binds to the CRISPR-associated protein, or (ii) a combination of a CRISPR-RNA (crRNA) molecule that is configured to bind to the protospacer in the DNA target and a trans-activating crRNA (tracrRNA) molecule that binds to the CRISPR-associated protein.

10. A pharmaceutical composition which comprises the RNP delivery system of claim 1, in admixture or conjunction with a pharmaceutically suitable carrier.

11. A method for the treatment or prophylaxis of a condition caused in a human or in a non-human animal by a defect and/or a deficiency in a gene, or for RNA therapy, or for the treatment of a cancer, which comprises administering the RNP delivery system as claimed in claim 1 to the human or to the non-human animal.

12. An RNP delivery system as claimed in claim 1, for use as a medicament, for example, as a vaccine or for use in the treatment or prophylaxis of a condition caused in a human or a non-human animal by a defect and/or a deficiency in a gene, or for RNA therapy, or for the treatment of a cancer.

13. Use of an RNP delivery system as claimed in claim 1, for the manufacture of a medicament for the treatment or prophylaxis of a condition caused in a human or a non-human animal by a defect and/or a deficiency in a gene, or RNA therapy, or for the treatment of a cancer.

14. A method of making an RNP delivery system as claimed in claim 1, the method comprising the steps of:
(a) providing a ribonucleoprotein (RNP) complex comprising a CRISPR-associated protein and guide RNA;
(b) combining the ribonucleoprotein complex from step (i) with lipid component comprising a phospholipid and a cationic lipid; and
(c) adding a peptide containing (A) a polycationic nucleic acid-binding component, (B) a spacer element comprising two or more amino acid residues and (C) a cell surface receptor binding component to the mixture from step (ii) to form the RNP delivery system.

15. A method of editing a gene in mammalian cell comprising transfecting the cell with the RNP delivery system as claimed in claim 1.
